(19) United States
(12) Patent Application Publication
(10) Pub. No.: US 2026/0000524 A1
(43) Pub. Date: Jan. 1, 2026

(54) TECHNOLOGIES FOR NON-VISUAL MONITORING OF AN ORTHOPAEDIC SURGICAL PROCEDURE

(71) Applicant: DePuy Ireland Unlimited Company, Ringaskiddy (IE)

(72) Inventors: Jackson R. Heavener, Warsaw, IN (US); Sarah E. Radcliffe, Leeds (GB)

(21) Appl. No.: 19/224,142

(22) Filed: May 30, 2025

Related U.S. Application Data
(60) Provisional application No. 63/665,693, filed on Jun. 28, 2024.

(51) Int. Cl. A61F 2/46; A61B 17/00; A61B 17/16; A61B 34/00; A61B 34/20

(52) U.S. Cl. CPC A61F 2/461; A61B 17/1675; A61B 34/20; A61B 34/76; A61B 2017/00115; A61F 2002/4666

(57) ABSTRACT

Technologies for non-visual monitoring of an orthopaedic surgical procedure includes determining a difference between a measured or sensed value of an aspect of the orthopaedic surgical procedure and a target value of the aspect of the orthopaedic surgical procedure. A non-visual indicator is activated to produce a non-visual indication having a characteristic that is dependent on a magnitude of the difference between the measured or sensed value and the target value. In some embodiments, the aspect of the orthopaedic surgical procedure may include as a force established between a patient's tibia and femur. In other embodiments, the aspect the orthopaedic surgical procedure may include a location of a femoral broach relative to the patient's boney anatomy.

This application claims priority under 35 U.S.C. § 119 (e) to U.S. Provisional Patent Application No. 63/665,693, entitled “SYSTEM, DEVICE, AND METHOD FOR MONITORING FORCES OF A PATIENT'S KNEE JOINT DURING PERFORMANCE OF AN ORTHOPAEDIC SURGICAL PROCEDURE,” which was filed on Jun. 28, 2024, which is incorporated by reference in its entirety.

### TECHNICAL FIELD

The present disclosure relates generally to orthopaedic surgical devices and, more particularly, to systems, devices, and methods for monitoring forces of a patient's knee joint during the performance of an orthopaedic surgical procedure on the patient's knee joint.

### BACKGROUND

Joint arthroplasty is a well-known orthopaedic surgical procedure by which a diseased and/or damaged natural joint is replaced by a prosthetic joint, which may include one or more orthopaedic implants. For example, in a knee arthroplasty surgical procedure, a patient's natural knee joint is partially or totally replaced by a prosthetic knee joint. A typical prosthetic knee joint includes a tibial tray, a femoral component, and a polymer insert or bearing positioned between the tibial tray and the femoral component.

During a typical orthopaedic surgical procedure, a surgeon initially prepares the patient's bone(s) to receive the orthopaedic prosthesis. For example, in the case of a knee replacement orthopaedic surgical procedure, the surgeon may resect a portion of the patient's proximal tibia to which the tibia tray will be attached, a portion of patient's distal femur to which the femoral component will be attached, and/or a portion of the patient's patella to which the patella component will be attached. Prior to and/or during such procedures, the surgeon may attempt to plan for and/or actively balance or otherwise distribute the joint forces of the patient's joint in order to produce joint motion that is similar to the motion of a natural joint. To do so, in some cases, the surgeon may use surgical experience and manually “feel” for the appropriate joint force balance. Additionally or alternatively, the orthopaedic surgeon may use surgical instruments to measure such joint forces. Typically, such force measurement devices provide a visual feedback, which may include the measured force values, to the orthopaedic surgeon. However, monitoring both the patient's knee joint (e.g., monitoring the joint gap space) and the visual feedback during the orthopaedic surgical procedure can be challenging for the orthopaedic surgeon.

Hip arthroplasty is another type of orthopaedic surgical procedure in which an orthopaedic surgeon prepares the proximal femur and/or acetabulum of a patient to receive an orthopaedic prosthesis. For example, in regard to femoral preparation, the surgeon may ream the proximal end of a patient's femur to a desired depth to prepare the femur to receive a femoral head prosthesis. Again, the particular reaming depth may be based on the surgical experience. Alternatively, certain navigation systems may be used to help the orthopaedic surgeon monitor the reamed depth. However, such navigation systems typically provide only visual feedback to the surgeon, which can create challenges in monitoring both the patient's hip joint and the visual feedback.

### SUMMARY

According to an aspect of the present disclosure, an orthopaedic surgical device may include a tibial paddle, a sensor array, and a controller. The tibial paddle may be shaped to be positioned between a proximal tibia and distal femur of a patient's knee joint. The sensor array may be positioned in the tibial paddle and may include a plurality of sensors configured to generate sensor data indicative of a force between the patient's tibia and femur. The controller may be configured to receive the sensor data from the sensor array, determine a difference between the sensor data and a target force value, and activate the non-visual indicator to produce a non-visual indication having a characteristic that is dependent on a magnitude of the difference between the sensor data and the target force value.

In some embodiments, the controller may be configured to receive sensor data indicative of a force on a medial or a lateral side of the patient's knee joint. In such embodiments, the target force value may be equal to a previously measured force value of the other one of the medial or the lateral side of the patient's knee joint. Additionally, in some embodiments, the non-visual indicator may be embodied as an audible indicator. In such embodiments, the controller may be configured to activate the audible indicator to produce an audible indication having a characteristic that is dependent on the magnitude of the difference between the sensor data and the target force value. Additionally or alternatively, in some embodiments, the non-visual indicator may be embodied as a tactile indicator. In such embodiments, the controller may be configured to activate the tactile indicator to produce a tactile indication having a characteristic that is dependent on the magnitude of the difference between the sensor data and the target force value.

Additionally, in some embodiments, the controller may be configured to modify the characteristic of the non-visual indication as a function of the magnitude of the difference between the sensor data and the target force value. For example, in some embodiments, the non-visual indication may include a plurality of individual indications that are produced at an activation frequency. In such embodiments, the controller may be configured to modify the activation frequency of the plurality of individual indications. In some embodiments, for example, the non-visual indicator may be embodied as an audible indicator configured to produce, when activated, a plurality of individual audible indications at the activation frequency.

Additionally or alternatively, in some embodiments, the non-visual indication may be embodied as a non-visual indication having a signal frequency. In such embodiments, the controller may be configured to modify the signal frequency of the non-visual indication. In some embodiments, for example, the non-visual indicator may be embodied as an audible indicator configured to produce, when activated, an audible indication having the signal frequency.

In some embodiments, the controller may be configured to determine a difference between the sensor data and each of a plurality of target force values. In such embodiments, the controller may be further configured to activate the non-visual indicator to produce a non-visual indication having multiple characteristics, each of which is dependent on a magnitude of the difference between the sensor data and a different one of the plurality of target force values.

According to another aspect of the present disclosure, a method for monitoring joint force of a patient's knee joint during performance of an orthopaedic surgical procedure may include determining, by a sensor module located between a proximal tibia and a distal femur of the patient's knee joint, sensor data indicative of a force between the patient's tibia and femur; determining, by the sensor module, a difference between the sensor data and a target force value; and activating, by the sensor module, a non-visual indicator of the sensor module as a function of the difference between the sensor data and the target force value. The non-visual indicator, when activated, may produce a non-visual indication having a characteristic that is dependent on a magnitude of the difference between the sensor data and the target force value.

In some embodiments, determining sensor data indicative of the force between the patient's tibia and femur may include determining sensor data indicative of a force on a medial or a lateral side of the patient's knee joint. In such embodiments, the target force value may be equal to a previously measured force value of the other one of the medial or the lateral side of the patient's knee joint. Additionally, in some embodiments, activating the non-visual indicator may include activating an audible indicator as a function of the difference between the sensor data and the target force value. Additionally or alternatively, in some embodiments, activating the non-visual indicator may include activating a tactile indicator as a function of the difference between the sensor data and the target force value.

Additionally, in some embodiments, activating the non-visual indicator of the sensor module may include modifying the characteristic of the non-visual indication as a function of the magnitude of the difference between the sensor data and the target force value. For example, in some embodiments, the non-visual indication may include a plurality of individual indications that are produced at an activation frequency. In such embodiments, modifying the characteristic of the non-visual indication may include modifying the activation frequency of the plurality of individual indications. Additionally or alternatively, the non-visual indication may include a non-visual indication have a signal frequency. In such embodiments, modifying the characteristic of the non-visual indication may include modifying the signal frequency of the non-visual indication.

In some embodiments, determining the difference between the sensor data and the target force value may include determining a difference between the sensor data and each of a plurality of target force values. In such embodiments, activating the non-visual indicator may include activating the non-visual indicator as a function of the differences between the sensor data and the plurality of target force values. The non-visual indicator, when activated, may produce a non-visual indication having multiple characteristics, each of which is dependent on a magnitude of the difference between the sensor data and a different one of the plurality of target force values.

According to yet a further aspect of the present disclosure, an orthopaedic surgical device may include a tibial paddle, a sensor array positioned in the tibial paddle, an audible indicator, and a controller. The tibial paddle may be shaped to be positioned between a proximal tibia and distal femur of a patient's knee joint. The sensor array may include a plurality of sensors configured to generate sensor data indicative of a force on a medial or a lateral side of the patient's knee joint. The audible indicator may be configured to generate an audible indication. Additionally, the controller may be configured to receive the sensor data from the sensor array, determine a difference between the sensor data and a target force value that is equal to a previously measured force value of the other one of the medial or the lateral side of the patient's knee joint, and activate the audible indicator to produce the audible indication having a characteristic that is dependent on a magnitude of the difference between the sensor data and the target force value.

In some embodiments, the audible indication may include a series of individual audible indications. In such embodiments, the controller may be configured to activate the audible indicator to produce the series of individual audible indications at an activation frequency that is dependent on the magnitude of the difference between the sensor data and the target force value.

According to another aspect, a surgical navigation system may include a tracking system, a non-visual indicator, and a controller. The tracking system may be configured to determine a present location of a surgical instrument relative to a femur of a patient. The controller may be configured to determine a present location of a femoral broach attached to the surgical instrument based on the location of the surgical instrument, determine a difference between the present location of the femoral broach and a reference location indicative of a target depth of the femoral broach in the femur of the patient, and activate the non-visual indicator to produce a non-visual indication having a characteristic that is dependent on a magnitude of the difference between the location of the femoral broach and the reference location of the target depth of the femoral broach in the femur of the patient.

In some embodiments, the non-visual indicator may be embodied as an audible indicator. In such embodiments, to activate the non-visual indicator may include to activate the audible indicator to produce an audible indication having a characteristic that is dependent on the magnitude of the difference between the location of the femoral broach and the reference location of the target depth of the femoral broach in the femur of the patient. Additionally or alternatively, in some embodiments, the non-visual indicator may be embodied as a tactile indicator. In such embodiments, to activate the non-visual indicator may include to activate the tactile indicator to produce an tactile indication having a characteristic that is dependent on the magnitude of the difference between the location of the femoral broach and the reference location of the target depth of the femoral broach in the femur of the patient.

Additionally, in some embodiments, to activate the non-visual indicator may include to modify the characteristic of the non-visual indication as a function of the magnitude of the difference between the location of the femoral broach and the reference location of the target depth of the femoral broach in the femur of the patient. For example, the non-visual indication may include a plurality of individual indications that are produced at an activation frequency, and to modify the characteristic of the non-visual indication may include to modify the activation frequency of the plurality of individual indications. In such embodiments, the non-visual indicator may be embodied as an audible indicator configured to produce, when activated, a plurality of individual audible indications at the activation frequency.

Additionally or alternatively, the non-visual indication may be embodied as a non-visual indication having a signal frequency, and to modify the characteristic of the non-visual indication may include to modify the signal frequency of the non-visual indication. In such embodiments, the non-visual indicator may be embodied as an audible indicator configured to produce, when activated, an audible indication having the signal frequency.

In some embodiments, the controller may be further configured to determine a present alignment of the femoral broach relative to the femur of the patient based on the location of the surgical instrument and determine a difference between the present alignment of the femoral broach and a reference alignment. In such embodiments, to activate the non-visual indicator may include to activate the non-visual indicator to produce a non-visual indication having multiple characteristics, wherein (i) a first characteristic of the multiple characteristics is dependent on the magnitude of the difference between the location of the femoral broach and the reference location of the target depth of the femoral broach in the femur of the patient and (ii) a second characteristic of the multiple characteristics is dependent on the magnitude of the difference between the present alignment of the femoral broach and a reference alignment. Additionally, in some embodiments, the reference alignment may be embodied as a target amount of femoral anteversion or femoral inclination.

According to yet a further aspect of the present disclosure, a method for monitoring operation of a surgical instrument during performance of an orthopaedic surgical procedure may include determining, by a surgical navigation system, a present location of a surgical instrument relative to a femur of a patient; determining, by the surgical navigation system, a present location of a femoral broach attached to the surgical instrument based on the location of the surgical instrument; determining, by the surgical navigation system, a difference between the present location of the femoral broach and a reference location indicative of a target depth of the femoral broach in the femur of the patient; and activating, by the surgical navigation system, a non-visual indicator to produce a non-visual indication having a characteristic that is dependent on a magnitude of the difference between the location of the femoral broach and the reference location of the target depth of the femoral broach in the femur of the patient.

In some embodiments, activating the non-visual indicator may include activating an audible indicator to produce an audible indication having a characteristic that is dependent on the magnitude of the difference between the location of the femoral broach and the reference location of the target depth of the femoral broach in the femur of the patient. Additionally or alternatively, activating the non-visual indicator may include activating a tactile indicator to produce an tactile indication having a characteristic that is dependent on the magnitude of the difference between the location of the femoral broach and the reference location of the target depth of the femoral broach in the femur of the patient.

Additionally, in some embodiments, activating the non-visual indicator may include modifying the characteristic of the non-visual indication as a function of the magnitude of the difference between the location of the femoral broach and the reference location of the target depth of the femoral broach in the femur of the patient. For example, the non-visual indication may include a plurality of individual indications that are produced at an activation frequency, and modifying the characteristic of the non-visual indication may include modifying the activation frequency of the plurality of individual indications. In such embodiments, for example, the non-visual indicator may be embodied as an audible indicator configured to produce, when activated, a plurality of individual audible indications at the activation frequency.

In some embodiments, the non-visual indication may include a non-visual indication having a signal frequency, and modifying the characteristic of the non-visual indication may include modifying the signal frequency of the non-visual indication. In such embodiments, for example, the non-visual indicator may be embodied as an audible indicator configured to produce, when activated, an audible indication having the signal frequency.

Additionally, in some embodiments, the method may further include determining, by the surgical navigation system, a present alignment of the femoral broach relative to the femur of the patient based on the location of the surgical instrument; and determining, by the surgical navigation system, a difference between the present alignment of the femoral broach and a reference alignment. In such embodiments, activating the non-visual indicator may include activating the non-visual indicator to produce a non-visual indication having multiple characteristics, wherein (i) a first characteristic of the multiple characteristics is dependent on the magnitude of the difference between the location of the femoral broach and the reference location of the target depth of the femoral broach in the femur of the patient and (ii) a second characteristic of the multiple characteristics is dependent on the magnitude of the difference between the present alignment of the femoral broach and a reference alignment. Additionally, in some embodiments, the reference alignment may be embodied as a target amount of femoral anteversion or femoral inclination.

### BRIEF DESCRIPTION OF THE DRAWINGS

The detailed description particularly refers to the following figures, in which:

FIG. 1 is a simplified diagram of an embodiment of a system for monitoring forces of a patient's knee joint during performance of an orthopaedic surgical procedure;

FIG. 2 is a perspective view of an embodiment of a sensor module of the system of FIG. 1;

FIG. 3 is an exploded, perspective view of the sensor module of FIG. 2;

FIG. 4 is a simplified diagram of an embodiment of a sensor array of the sensor module of FIG. 2;

FIG. 5 is a simplified diagram of another embodiment of the sensor array of the sensor module of FIG. 2;

FIG. 6 is a simplified block diagram an embodiment of a control circuit of the sensor module of FIG. 2;

FIGS. 7 and 8 is a simplified flow diagram of an embodiment of a method for monitoring forces of a patient's knee joint during performance of an orthopaedic surgical procedure, which may be executed by the sensor module of FIG. 2;

FIG. 9 is a simplified illustration of a graphical user interface that may be displayed on a display module of the system of FIG. 1;

FIG. 10 is a perspective view of a patient's joint in extension during an orthopaedic surgical procedure using the sensor module of FIG. 2;

FIG. 11 is a perspective view of the patient's joint in flexion during an orthopaedic surgical procedure using the sensor module of FIG. 2;

FIG. 12 is a simplified block diagram of a surgical navigation system configured to monitor operation of a surgical instrument during the performance of an orthopaedic surgical procedure;

FIGS. 13 and 14 is a simplified flow diagram of an embodiment of a method for monitoring operation of a surgical instrument during the performance of an orthopaedic surgical procedure, which may be executed by the surgical navigation system of FIG. 12;

FIG. 15 is a simplified diagram of a femoral broaching surgical instrument during the performance of a femoral reaming surgical procedure; and

FIG. 16 is another simplified diagram of the femoral broaching surgical instrument of FIG. 15 during the performance of the femoral reaming surgical procedure.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

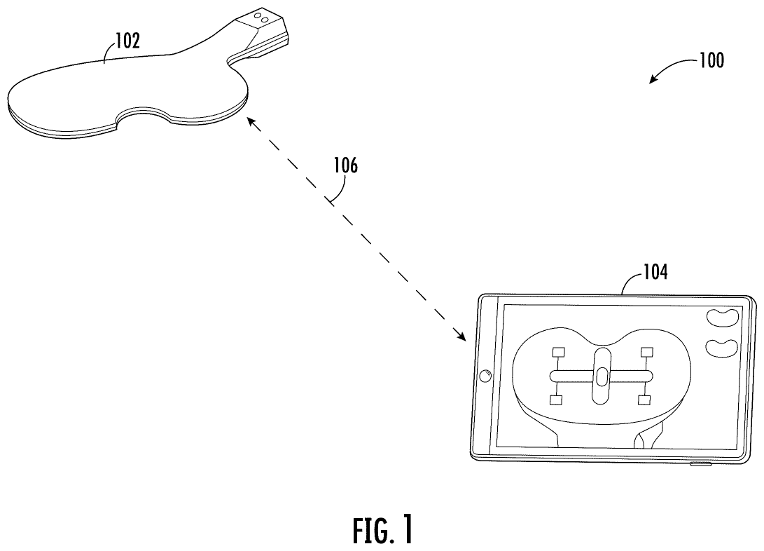

Referring now to FIG. 1, in an illustrative embodiment, a system 100 for monitoring forces of a patient's knee joint during performance of an orthopaedic surgical procedure includes a sensor module 102 and a remote display module 104, which is illustratively embodied as a hand-held remote display module. In other embodiments, the system 100 may include other devices such as a remote computer system, a computer assisted surgery system (CAOS), a robotic-assisted surgery system, and/or other orthopaedic devices and/or systems.

As discussed in more detail below, during performance of the orthopaedic surgical procedure, the sensor module 102 is configured to be inserted into a patient's knee joint and provide non-visual feedback of the joint forces of the medial and/or lateral joint forces of the patient's knee joint. For example, as discussed below, the sensor module 102 may provide an audible or tactile indication (along with a visual indication, in some embodiments) to the orthopaedic surgeon as to the relative difference between a measured force value and a target force value. For example, the measured force value may be indicative of a force value measured on the medial or lateral side of the patient's knee joint, while the target force value may be a force value previously measured on the opposite side of the patient's knee joint. The audible and/or tactile indication may change or otherwise be modified as the magnitude of the difference between the measured force value and the target force value changes, which may provide non-visual feedback to the orthopaedic surgeon regarding how close the measured force value is to the target force value. For example, the sensor module 102 may be configured to generate a series of audible beeps, the frequency of which (e.g., the activation frequency of the series of the audible beeps and/or the signal frequency of the audible beep itself) changes as the difference between the measured force value and the target force value changes. In an illustrative embodiment, for example, the frequency of a series of audible beeps may increase (i.e., the individual beeps may be generated closer in temporal proximity to each other) as the measured force value nears the target force value. Similarly, the frequency of a series of tactile vibrations may increase as the measured force value nears the target force value. In this way, the orthopaedic surgeon may be updated as to the measured and target force values while viewing and/or manipulating the patient's knee join without the requirement of averting their gaze to view visual indications provided by, for example, the display module 104.

The sensor module 102 may be communicatively coupled to the remote display module 104 via a wireless communication link 106 (e.g., a Bluetooth communication link) and configured to transmit data including, for example, the measured force values to the remote display module 104. The display module 104 may be embodied as any type of hand-held computer device capable of displaying information to the orthopaedic surgeon during performance of the orthopaedic surgical procedure. For example, as discussed in more detail below, the display module 104 may be configured to display a graphical user interface that includes the measured and/or target force values. Although illustrated in FIG. 1 as a hand-held remote display module 104, the display module 104 may be embodied as a typical computer, such as a desktop computer, a CAOS computer, a robotic-assisted surgery computer, and/or other computer device or system in other embodiments. It should be appreciated, however, that the orthopaedic surgeon may rely on the non-visual indications provided by the sensor module 102 instead of or in addition to any visual indications (e.g., visual force values) displayed on the display module 104.

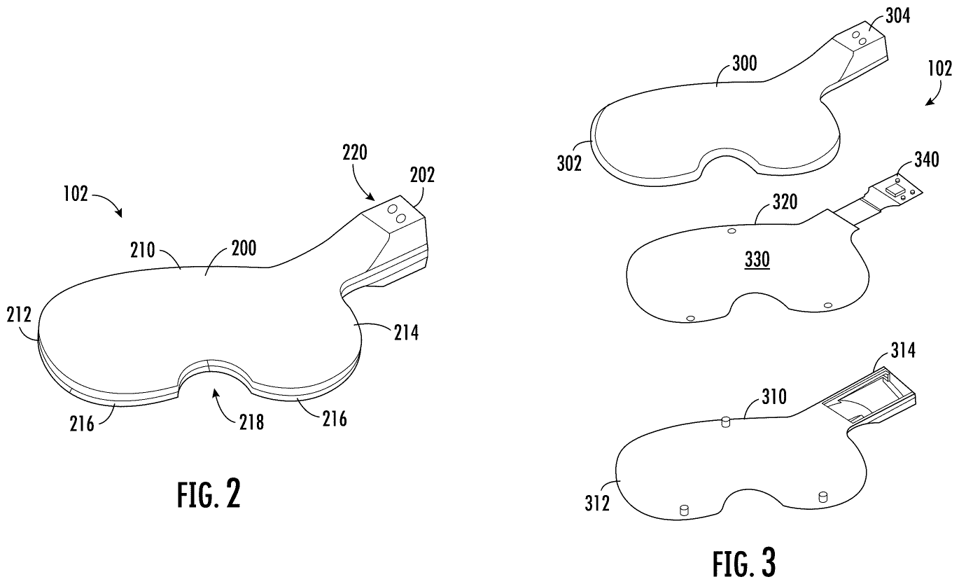

Referring now to FIG. 2, the illustrative sensor module 102 includes a tibial paddle 200 and a circuit housing 202 coupled to the tibial paddle 200. As illustrated in FIG. 2, the tibial paddle 200 is sized and shaped to be positioned in a knee joint of the patient. However, in other embodiment, the sensor module 102 may be configured to be used with other joints of the patient and, in such embodiments, the tibial paddle 200 may have a different sensor housing shape configured to be inserted into a corresponding joint of the patient. For example, the tibial paddle 200 may be embodied, instead, as an acetabular sensor housing configured to be inserted into a hip joint of the patient.

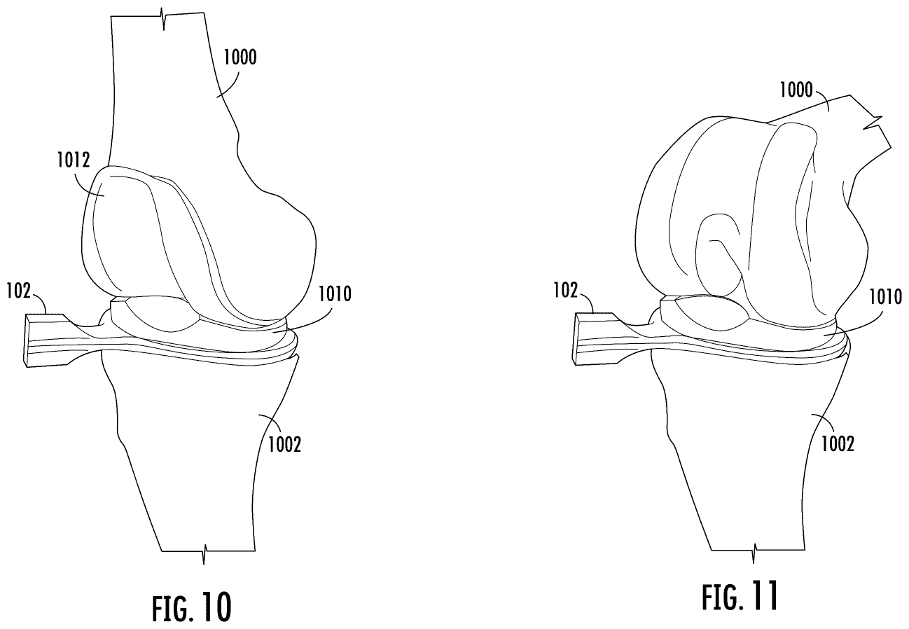

In use, the tibial paddle 200 is configured to be positioned on a proximal resected or non-resected plateau of a patient's resected tibia (see, e.g., FIGS. 10 and 11). To do so, the tibial paddle 200 may be placed in contact with the patient's tibia or may be placed on an intervening platform or other member. Additionally, the sensor module 102 may be used on the patient's left or right knee. For example, the sensor module 102 may be used on a patient's left knee via a medial surgical approach wherein the tibial paddle 200 is inserted into the patient's left knee joint via a medial capsular incision. In such position, the circuit housing 202 extends out of the medial capsular incision. Alternatively, by simply flipping or turning over the sensor module 102, the module 102 may be used on the patient's left knee via a lateral surgical approach wherein the tibial paddle 200 is inserted into the patient's left knee joint via a lateral capsular incision. Again, in such position, the circuit housing 202 extends out of the lateral capsular incision.

As such, it should be appreciated that sensor module 102 may be used on the patient's left or right knee using a medial or lateral surgical approach. For clarity of description, the sensor module 102 and the system 100 are described below with reference to an orthopaedic surgical procedure using a medial surgical approach (i.e., using a medial capsular incision to access the patient's joint). However, it should be appreciated that such description is equally applicable to lateral surgical approach procedures. As such, some structures are described using particular anatomical references (e.g., lateral and medial) with the understanding that such references would be flipped or switched when the sensor module 102 is used in a lateral surgical approach procedure. For example, a “medial side” of the tibial paddle 200 becomes a “lateral side” of the tibial paddle 200 when used in a lateral surgical approach procedure.

As shown in FIG. 2, the illustrative tibial paddle 200 is planar (within manufacturing tolerances) and has a shape generally corresponding to the shape of the orthopaedic prosthesis to be implanted in the patient. For example, in the illustrative embodiment, the tibial paddle 200 has a shape generally corresponding to a tibial prosthesis of a particular size. However, in other embodiments in which the sensor module 102 is configured for use in other joints, the paddle 200 (or sensor housing) may have a shape generally corresponding to other types of orthopedic prostheses such as a hip prosthesis, a shoulder prosthesis, an ankle prosthesis, a spine prosthesis, or a patella prosthesis.

The illustrative tibial paddle 200 includes a curved anterior side 210, a curved lateral side 212, a curved medial side 214, and a curved posterior side 216, each shaped to approximate the shape a tibial bearing of an orthopaedic knee prosthesis. Again, as discussed above, the lateral side 212 and the medial side 214 are lateral and medial sides, respectively, in those embodiments wherein the sensor module 102 is used in a lateral surgical approach procedure. The posterior side 216 includes a posterior notch 218 to allow the tibial paddle 200 to be positioned around the soft tissue of the patient's joint such as the posterior cruciate ligament. Additionally, in some embodiments, the posterior notch 218 may also be configured to provide a mount for other surgical devices such as a trial post for rotating mobile bearing trials. Further, in some embodiments, the posterior notch 218 may be extended or otherwise have other configurations so as to provide a mount for other orthopaedic surgical devices such as fixed and/or mobile tibial trials or the like.

The overall size of the tibial paddle 200 may be selected based on the particular anatomical structure of the patient. For example, in some embodiments, the tibial paddle 200 may be provided in various sizes to accommodate patients of varying sizes. It should be appreciated that the general shape and size of the paddle 200 is designed and selected such that the paddle 200 does not significantly overhang with respect to the associated bony anatomy of the patient nor adversely impinge the surrounding soft tissue.

In the illustrative embodiment, the circuit housing 202 includes a user interface 220, which may be embodied as a one or more buttons or other input devices. The user interface 220 allows the orthopaedic surgeon to provide input to the sensor module 102 or otherwise control the sensor module 102. For example, the orthopaedic surgeon may operate the user interface 220 (e.g., select one of the user buttons) to “lock” a presently measured force value or otherwise perform a “peak and hold” value acquisition such that that measured force value can be used as the target measured force value. In this way, the orthopaedic surgeon may measure the force value on the medial or lateral side of the patient's knee joint, lock the measured force value as the target force value, and subsequently measure the other side of the patient's knee joint using the previously measured force value as the target force value to thereby assist in the balancing of the medial and lateral joint forces. As discussed above, as the difference between presently measured and target force values is reduced, the non-visual indication may change to alert the orthopaedic surgeon that the measured force value is approaching the target force value.

The circuit housing 202 extends from a side of the tibial paddle 200 as shown in FIG. 2. In the illustrative embodiment, the circuit housing 202 extends from the medial side 214 (which is a lateral side when the sensor module 102 is used in a lateral surgical approach procedure). It should be appreciated that because the circuit housing 202 extends from a side of the paddle 200, the tibial paddle 200 may be positioned in a knee joint of a patient without the need to sublux or evert the patient's patella. That is, the tibial paddle 200 may be properly positioned between the patient's proximal tibia and distal femur with the patient's patella in the natural position.

In the illustrative embodiment of FIG. 2, the tibial paddle 200 and the circuit housing 202 are substantially monolithic in structure. Each of the tibial paddle 200 and the circuit housing 202 is illustratively formed from a bio-compatible material. For example, the tibial paddle 200 and the circuit housing 202 may be formed from a bio-compatible plastic or polymer. In some embodiments, the sensor module 102 is configured for single-usage and, as such, is provided in a sterile form. For example, the sensor module 102 may be provided in a sterile packaging. However, the sensor module 102 may be configured to be reusable via an autoclaving procedure or the like.

Referring now to FIG. 3, the illustrative sensor module 102 includes an upper housing 300 and a lower housing 310, which are coupled to each other. In some embodiments, the upper housing 300 and the lower housing 310 are mirror images of each other. The upper housing 300 includes an upper tibial paddle housing 302 and an upper circuit housing 304. Similarly, the lower housing 310 includes a lower tibial paddle housing 312 and a lower circuit housing 314.

The sensor module 102 also includes a sensor component 320, which is configured to positioned between, and housed in, the upper housing 300 and the lower housing 310. The sensor component 320 includes a sensor array 330, which is positioned in the tibial paddle 200, and a control circuit 340 communicatively coupled to the sensor array 330. The sensor array 330 is “sandwiched” between the upper housing 300 and the lower housing 310. However, the upper housing 300 and the lower housing 310 are spaced apart to allow the sensor array 330 to be compressed by the joint force applied to the tibial paddle 200.

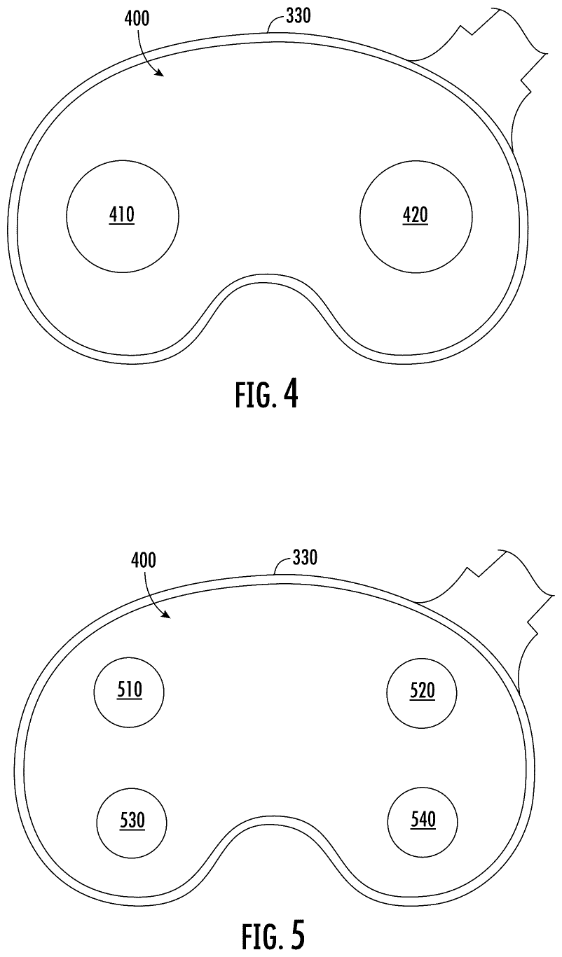

Referring now to FIG. 4, the illustrative sensor array 330 includes a plurality of pressure sensors or sensor elements 400 configured to generate sensor signals indicative of the joint force applied to the sensor array 330. The pressure sensors 400 may be embodied as any type of sensor capable of generating sensor data indicative of a force applied to the sensor. In the illustrative embodiment, for example, the pressure sensors 400 are embodied as capacitive pressure sensors. As shown in FIG. 4, in some embodiments, the sensor array 330 may include one or more lateral sensors 410 configured to sense a lateral joint force of the patient's knee joint and one or more medial sensors 420 configured to sense a medial joint force of the patient's knee joint. That is, the sensor array 330 may be configured to measure medial and lateral joint force only. Alternatively, in other embodiments, the sensor array 330 may include additional sensors to improve the granularity of the joint force measurements. For example, as shown in FIG. 5, the sensor array 330 may include one or more anterior-lateral sensors 510 configured to sense an anterior-lateral joint force of the patient's knee joint, one or more anterior-medial sensors 520 configured to sense an anterior-medial joint force of the patient's knee joint, one or more posterior-lateral sensors 530 configured to sense an posterior-lateral joint force of the patient's knee joint, and one or more posterior-medial sensors 540 configured to sense an posterior-medial joint force of the patient's knee joint. In such embodiments, the sensor module 102 is configured to measure and determine independent anterior and posterior joint force values on each other medial and lateral side, which increases the granularity at which the orthopaedic surgeon may balance the patient's knee joint.

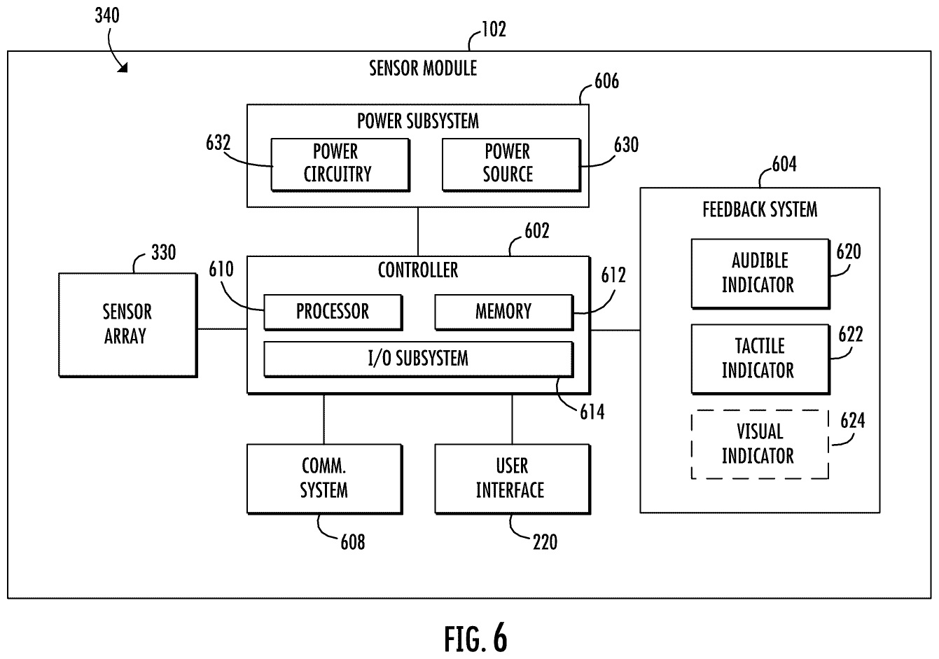

Referring now to FIG. 6, an illustrative embodiment of the control circuit 340 of the sensor module 102 includes a controller 602, a feedback system 604, a power subsystem 606, a communication system 608, the user interface 220, and the sensor array 330. In other embodiments, however, the control circuit 340 may include additional or other electronic devices and/or circuit.

The controller 602 may be embodied as any type of controller, functional block, digital logic, or other component, device, circuitry, or collection thereof capable of performing the functions described herein. In illustrative embodiment, the controller 602 includes a processor 610, a memory 612, and an input/output (I/O) subsystem 614. The processor 610 may be embodied as any type of processor capable of performing the functions described herein. For example, the processor 610 may be embodied as a single or multi-core processor(s), digital signal processor, microcontroller, or other processor or processing/controlling circuit. Similarly, the memory 612 may be embodied as any type of volatile and/or non-volatile memory or data storage capable of performing the functions described herein. In operation, the memory 612 may store various data and software used during operation of the sensor module 102 such as executable firmware or software, programs, libraries, and drivers, which may be executed or otherwise used by the processor 610.

The processor 610 and memory 612 are communicatively coupled to other components of the control circuit 340 via the I/O subsystem 614, which may be embodied as circuitry and/or components to facilitate input/output operations between the controller 602 (e.g., the processor 610 and the memory 612) and the other components of the sensor module 102. For example, the I/O subsystem 614 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 614 may form a portion of a system-on-a-chip (SoC) and be incorporated, along with the processor 610 and the memory 612, and other components of the sensor module 102, on a single integrated circuit chip. Additionally, in some embodiments, the memory 612, or portions of the memory 612, may be incorporated into the processor 610.

The feedback system 604 may be embodied as, or otherwise include, one or more non-visual indicators configured to provide a non-visual indication indicative of a difference between the presently measured force value of the patient's knee joint and the target force value. In the illustrative embodiment, the non-visual indication may have a characteristic (e.g., activation frequency, signal frequency, volume, etc.) that is dependent on a magnitude of the difference between the sensor data and the target force value. For example, the feedback system 604 may include one or more audible indicators 620 (e.g., a speaker) configured to generate, when activated, an audible indication having a characteristic (e.g., activation frequency, signal frequency, volume, etc.) that is dependent on a magnitude of the difference between the sensor data and the target force value. Additionally or alternatively, the feedback system 604 may include one or more tactile indicators 622 (e.g., a vibration motor) configured to generate, when activated, an tactile indication having a characteristic (e.g., activation frequency, signal frequency, vibration intensity, etc.) that is dependent on a magnitude of the difference between the sensor data and the target force value. In some embodiments, the feedback system 604 may also include one or more visual indicators 624 (e.g., a light or light emitting diode) in addition to the non-visual indicators discussed above. As discussed below, the visual indicators 624 may be used in conjunction with the non-visual indicators to provide an additional visual indication indicative of the difference between the presently measured force value of the patient's knee joint and the target force value.

The power subsystem 606 is configured to provide power to the other components of the control circuit 340. In the illustrative embodiment, the power subsystem 606 includes a power source 630 and power circuitry 632. The power source 630 may be embodied as any type of power source capable of providing an amount of power to the sensor module 102. For example, in some embodiments, the power source 630 may be embodied as a battery, which may be rechargeable. The power circuitry 632 may be embodied as any type of electrical device, component, or circuitry configured to manage and control the distribution of the power from the power source 630 to the other electrical components of the sensor module 102. For example, in some embodiments, the power circuitry 632 may convert the power provided by the power source 630 to a power level usable by the various other components of the sensor module 102.

The communication system 608 may be embodied as any type of communication circuit, device, or collection thereof, capable of enabling communications between the sensor module 102 and the remote display module 104 and/or other devices of the system 100. To do so, the communication system 608 may be configured to use any one or more communication technologies (e.g., wireless or wired communications) and associated protocols (e.g., Bluetooth®, Wi-Fi®,Ethernet WiMAX, LTE, 5G, etc.) to effect such communication.

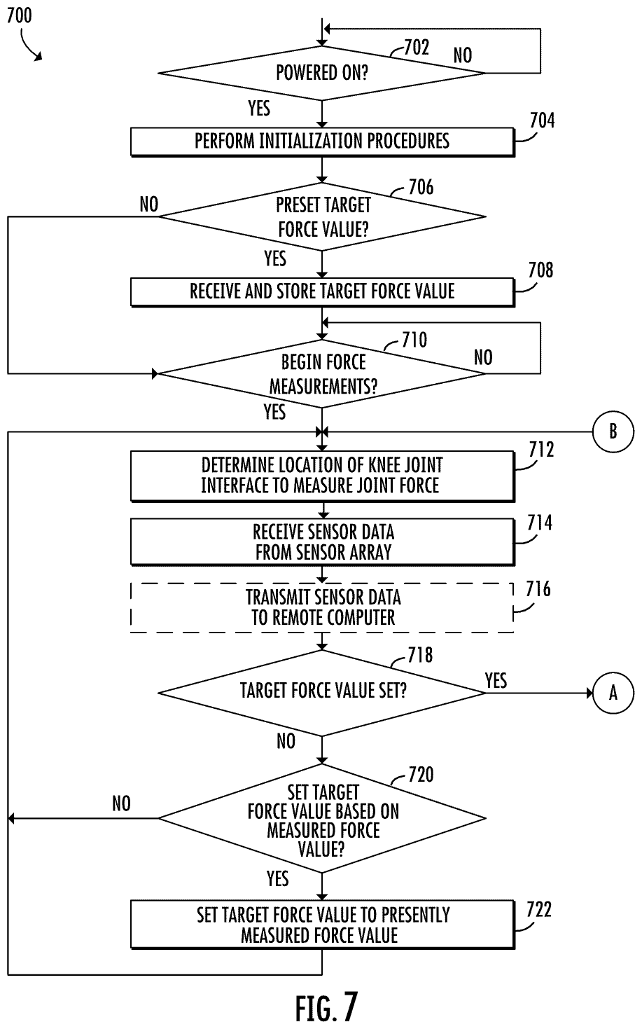

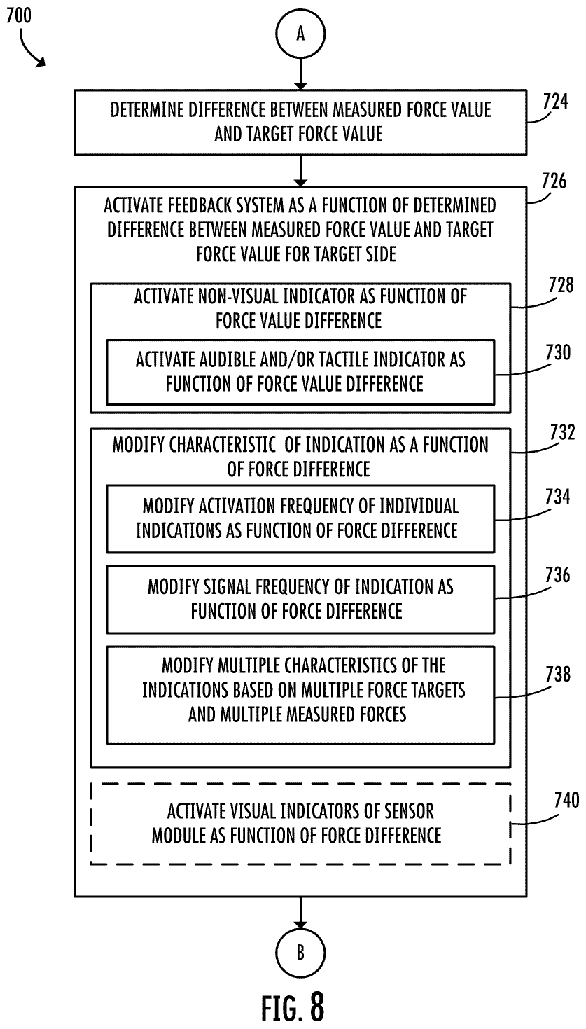

Referring now to FIGS. 7 and 8, in use, the sensor module 102 (e.g., the controller 602 of the control circuit 340) may be configured to execute a method 700 for monitoring joint force of a patient's knee joint during performance of an orthopaedic surgical procedure. The method 700 begins with block 702 in which the sensor module 102 determines whether the module 102 has been powered on. For example, the orthopaedic surgeon may power on the sensor module 102 via actuation of one of the buttons of the user interface 220. If the sensor module 102 determines that the orthopaedic surgeon has powered on the sensor module 102, the method 700 advances to block 704 in which the sensor module 102 performs various initialization procedures. For example, the sensor module 102 may determine an offset for the sensor array 330 so as to “zero out” the individual sensors of the sensor array 330.

In block 706, the sensor module 102 determines whether the orthopaedic surgeon desires to preset a target force value. For example, in some embodiments, the orthopaedic surgeon may operate the user interface 220 and/or the remote display module 104 to enter a target force value. In such situations, the orthopaedic surgeon may, for example, attempt to balance each of the medial and lateral side to the preset target force value, instead of balancing one side to the other side. Regardless, if the orthopaedic surgeon decides to supply a target force value, the method 700 advances to block 708 in which the sensor module 102 receives and stores the target force value. For example, the sensor module may store the received, preset target force value in the memory 612.

After the sensor module 102 has received and stored the target force value in block 708 or if the orthopaedic surgeon does not desire to preset the target force value in block 706, the method 700 advances to block 710. In block 710, the sensor module 102 determines whether to begin force measurements. For example, in some embodiments, the sensor module 102 may be configured to begin measuring the force between the patient's proximal tibia and distal femur in response to selection of a corresponding button of the user interface 220 by the orthopaedic surgeon.

If the sensor module 102 determines that force measurement is to begin in block 710, the method 700 advances to block 712 in which the sensor module 102 determines which location of the knee joint interface is to be measured. For example, the sensor module 102 may be instructed to measure the medial side or the lateral side. Alternatively, in embodiments in which the sensor array 300 includes more granular sensors as shown in FIG. 5, the sensor module 102 may measure the anterior-medial side, the posterior-medial side, the anterior-lateral side, or the posterior-lateral side. To do so, the orthopaedic surgeon may actively select the joint location via the user interface 220 (e.g., by cycling through the options) or select the joint location using the remote display module 104. As discussed in more detail below, the orthopaedic surgeon may change the location of the knee joint being measured after setting the target force value to a presently measured force value.

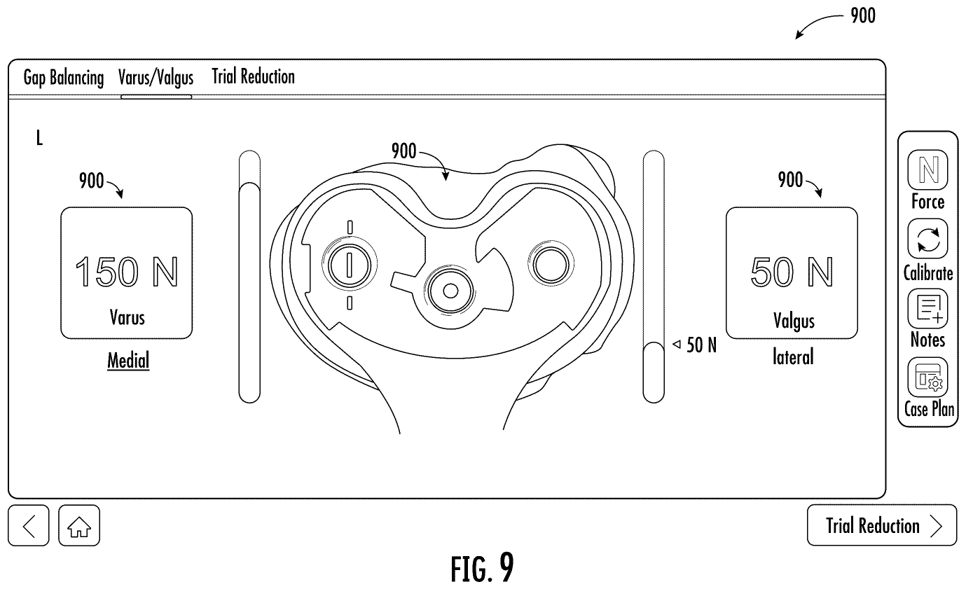

Regardless, after the sensor module 102 has determined which location of the knee joint to begin measuring the joint forces, the method 700 advances to block 714. In block 714, the controller 602 of the control circuit 340 receives sensor data from the sensor array 330. That is, the control circuit 340 receives sensor data indicative of a force sensed between the patient's tibia and femur. The particular format and/or structure of the sensor data may be dependent upon, for example, the particular pressure sensors of the sensor array 330. It should be appreciated that the controller 602 may receive sensor data from each of the sensors of the sensor array 330, even for locations not actively measured. In such embodiments, the controller 602 may ignore or otherwise not actively monitor the sensor data from joint locations not being measured based on the determination performed in block 712. Additionally, in some embodiments in block 716, the sensor module 102 may transmit the sensor data received in block 714 to the remote display module 104 for display thereon as discussed below in regard to FIG. 9.

Subsequently, in block 718, the sensor module 102 determines whether a target force value has been set. As discussed above in regard to block 706, the orthopaedic surgeon may preset the target force value. Alternatively, the orthopaedic surgeon may set the target force value intraoperatively to be equal to a presently measured force value. For example, the orthopaedic surgeon may set the target force value equal to a measured force value of the medial side of the patient' knee joint and monitor the measured force value of the lateral side by subsequently selecting the lateral side in block 712. Alternatively, the orthopaedic surgeon may set the target force value equal to a measured force value of the lateral side of the patient' knee joint and monitor the measured force value of the medial side by subsequently selecting the lateral side in block 712.

As such, if the sensor module 102 determines that a target force value has not yet been set in block 718, the method 700 advances to block 720 in which the sensor module 102 determines whether to set the target force value to the presently measured force value. If so, the method 700 advances to block 722 in which the sensor module 102 sets the target force value to the presently measured force value. In this way, the target force value can be set to a “hold” or, in some embodiments, a “peak and hold” value. Again, the orthopaedic surgeon may instruct the sensor module 102 to set the target force value to the presently measured force value via the user interface 220.

After the sensor module 102 has set target force value to the present measured force value in block 722 or if the sensor module 102 determines not to set the target force value in block 720, the method 700 loops back to block 712 in which the sensor module 102 determines the location of the knee joint interface to measure. In this way, for example, the orthopaedic surgeon can initially measure the joint force on one side of the patient's knee, set the target force value to that measured joint force, and then switch to actively measuring the joint force of the other side of the patient's knee.

Referring back to block 718, if the sensor module 102 determines that a target value has already been set (e.g., in block 708 or block 722), the method 700 advances to block 724 of FIG. 8. In block 724, the sensor module 102 determines a difference between the presently measured force value and the target force value. For example, the sensor module 102 may compare the presently measured force value to the target force value. Subsequently, in block 726, the sensor module 102 activates the feedback system 604 as a function of the determined difference between the presently measured force value and the target force value. In particular, in block 728, the sensor module 102 may activate the non-visual indicators as a function of the determined difference between the presently measured force value and the target force value. For example, in block 730, the sensor module 102 may activate the audible indicator(s) 620 and/or the tactile indicator(s) 622 as a function of the difference between the measured and target force values. In doing so, a characteristic of the non-visual indication provided by the non-visual indicator(s) may be dependent on a magnitude of the difference between the measured force value and the target force value. For example, the activation frequency (i.e., the temporal spacing) of a series of non-visual indications and/or the signal frequency of the non-visual indication may be dependent on the magnitude of the determined difference. In this way, if the magnitude of the determined difference is relatively large, the activation frequency of the non-visual indications may be low (e.g., individual audible “beeps” may be spaced apart by a larger amount of time) and/or the signal frequency of the non-visual indication may be low (e.g., the audible “beep” may be generated at a low signal frequency). Conversely, if the magnitude of the determined difference is relatively small, the activation frequency of the non-visual indications may be high (e.g., individual audible “beeps” may be close together in time) and/or the signal frequency of the non-visual indication may be high (e.g., the audible “beep” may be generated at a high signal frequency). In this way, the orthopaedic surgeon receives non-visual feedback as a notification that the measured force value is approaching or moving away from the target force value, without the requirement to view displayed force values.

Additionally, in block 732, the sensor module 102 may change or modify the characteristic(s) of the non-visual indication as a function of or based on the magnitude of the difference between the measured force value and the target force value. That is, as the magnitude changes, the sensor module 102 may likewise modify or change the characteristic. For example in block 734, the sensor module 102 may modify the activation frequency of individual indications as a function of the magnitude of the determined difference (e.g., increase the activation frequency of audible “beeps” in response to the magnitude decreasing). Additionally or alternatively, in block 736, the sensor module 102 may modify the signal frequency of the indication as a function of the magnitude of the determined difference (e.g., increase the signal frequency of the audible “beep” in response to the magnitude decreasing).

In some embodiments, in block 738, the sensor module 102 may modify multiple characteristics of the non-visual indications based on multiple force targets and/or multiple measured force values. For example, the activation frequency of the non-visual indication may correspond to a medial-lateral difference and the signal frequency of the non-visual indication may correspond to an anterior-posterior difference in measured and target force values. In this way, the orthopaedic surgeon may balance multiple locations of the patient's knee joint contemporaneously with each other.

In some embodiments, in block 740, the sensor module 102 may also activate one or more visual indicators 624 of the sensor module 102 as a function of the determined force difference, in addition to the non-visual indicators. Furthermore, as discussed in more detail below in regard to FIG. 9, the remote display module 104 may also disclose the measured and/or target force values, which the orthopaedic surgeon may optionally review. Regardless, after the sensor module 102 has activated the feedback system 604 in block 726, the method 700 loops back to block 712 of FIG. 7 in which the sensor module 102 again determines the location of the patient's knee joint interface at which to measure the joint force. In this way, the orthopaedic surgeon can cycle through different knee joint regions to balance those regions to each other and/or to a target joint force value.

Referring now to FIG. 9, as discussed above, the sensor module 102 may be configured to transmit the measured and/or target joint force values to the display module 104. In such embodiments, the display module 104 may display a graphical user interface 900. The illustrative graphical user interface 900 includes an image 902 of the sensor module 102 bounded on either side by displays 904, 906 of the medial force value and the lateral force value, respectively. It should be appreciated that one of the displayed medial and lateral force values may be set as the target force value, while the other displayed force value is the presently measured force value. For example, in the illustrated embodiment, the lateral joint force may have been measured first to be 50 newtons (N), which has been set to the target force value. As such, the medial joint force value is presently being measured at 150 newtons (N), and the orthopaedic surgeon may perform various techniques to balance the medial joint force value toward the lateral joint force value. While doing so and as discussed above, the sensor module 102 will provide a non-visual indication having a characteristic that is dependent upon the magnitude of the difference between the measured medial joint force value and the target lateral joint force value. As the magnitude of that difference changes, the sensor module 102 modifies the characteristic such that the orthopaedic surgeon is notified as to the magnitude of the difference, without the need of viewing the graphical user interface 900 of the display module 104.

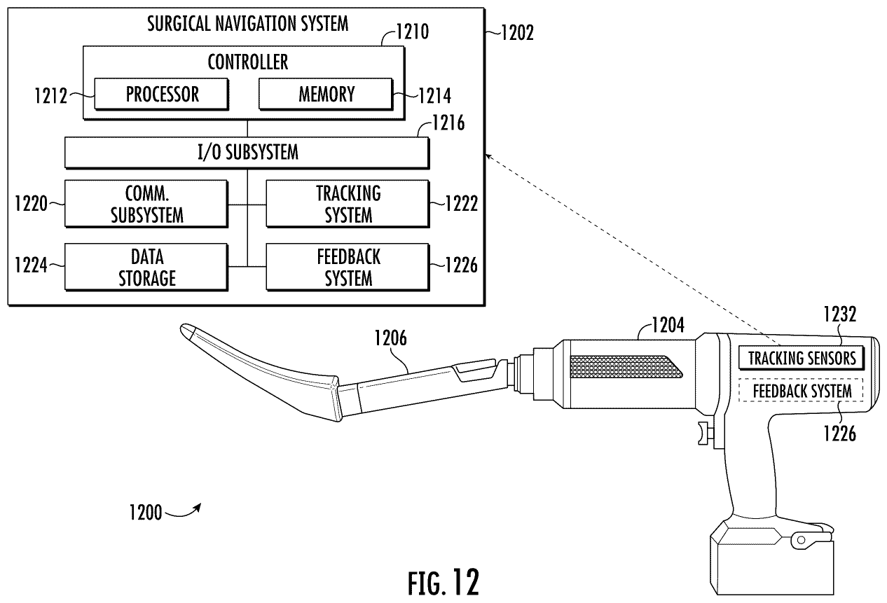

Referring now to FIGS. 10 and 11 and as discussed above, the sensor module 102 is configured to be inserted into the patient's knee joint between the patient's distal femur 1000 and proximal tibia 1002. In some embodiments, a tibial trial 1010 may be used with the sensor module 102. Additionally, while performing the joint force measurements, the orthopaedic surgeon may move the patient's knee joint through a range of flexion as shown in FIG. 11. The sensor module 102 may be used with a patient's resected or natural tibia and/or femur. For example, in the embodiment shown in FIGS. 10 and 11, the sensor module 102 is placed on a resected plateau of the patient's proximal tibia, and the distal end of the patient's femur 1000 has been replaced with an femoral prosthesis 1012. In other embodiments, however, the sensor module 102 may be used with a patient's un-resected tibia and/or femur. For example, in FIG. 12, the sensor module 102 is shown being used with a patient's natural distal femur.

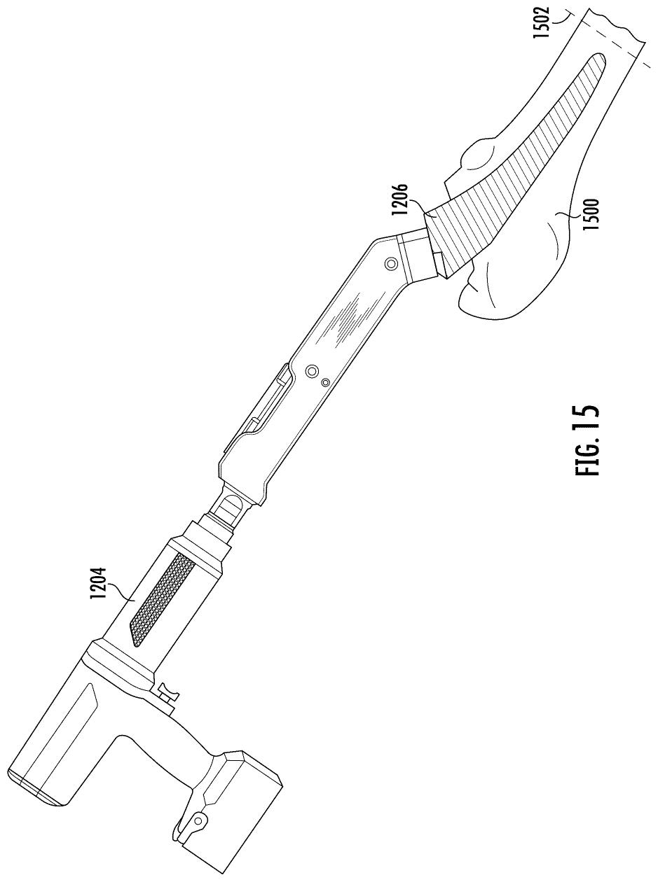

Referring now to FIG. 12, the above-described technologies for providing non-visual feedback to an orthopaedic surgeon during performance of an orthopaedic surgical procedure may be incorporated into systems configured to track the present location of surgical instruments to thereby provide non-visual indications of the relative positioning of such surgical instruments. For example, an illustrative system 1200 for monitoring operation of surgical instruments during performance of an orthopaedic surgical procedure includes a surgical navigation system 1202 and an orthopaedic surgical instrument 1204. The surgical navigation system 1202 is operable by an orthopaedic surgeon to complete an initial registration process to register the orthopaedic surgical instrument 1204 to the patient's bony anatomy (e.g., a femur 1500 of the patient as shown in FIG. 15). In doing so, the surgical navigation system 1202 may generate registration data indicative of the position of the orthopaedic surgical instrument 1204 in three-dimensional space relative to the patient's bony anatomy. Once so registered, the orthopaedic surgeon may then utilize the surgical navigation system 1202 to track the position of the orthopaedic surgical instrument 1204, relative to the patient's bony anatomy, during use of the orthopaedic surgical instrument 1204.

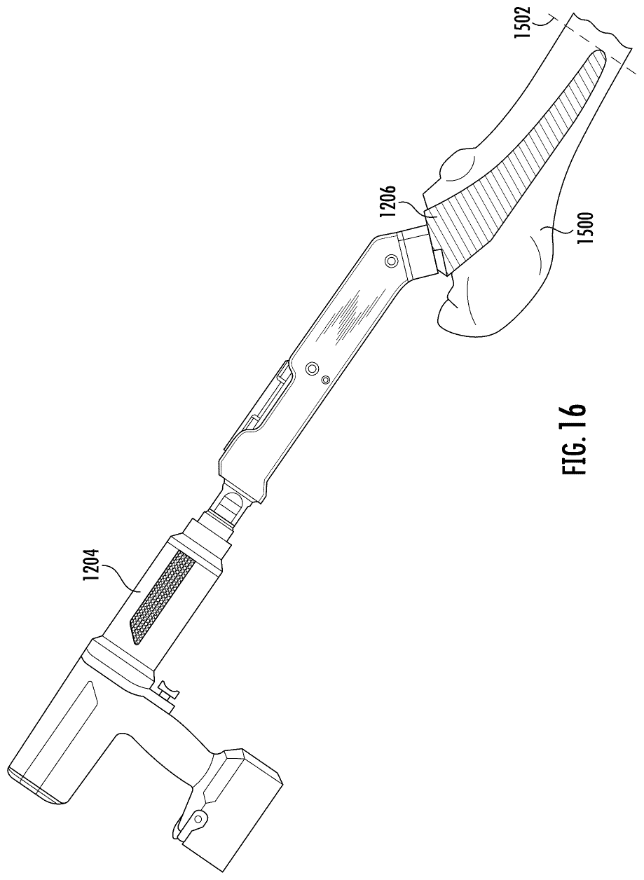

It should be appreciated that by tracking the position of the orthopaedic surgical instrument 1204, the surgical navigation system 1202 also tracks the position of any orthopaedic surgical tools attached to the registered orthopaedic surgical instrument 1204. For example, in the illustrative embodiment, the surgical navigation system 1202 is configured to track the location of a femoral broach 1206, attached to the surgical instrument 1204, relative to the patient's femur 1500 and provide non-visual feedback of the relative positioning. In doing so, as discussed in more detail below, the illustrative surgical navigation system 1202 is configured to compare the present location of the femoral broach 1206 to a reference location 1502 (see FIGS. 15 and 16) of the patient's femur 1500 indicative of a target depth of the femoral broach 1206 in the femur 1500 and determine a difference in those locations.

Additionally, the surgical navigation system 1202 provides the orthopaedic surgeon with a non-visual feedback indicative of the determined difference between the present position of the femoral broach 1206 and the reference location 1502 of the patient's femur 1500. For example, as discussed below, the surgical navigation system 1202 may provide an audible or tactile indication (along with a visual indication, in some embodiments) to the orthopaedic surgeon as to the relative difference between the present location of the femoral broach 1206 and the reference location 1502 of the patient's femur 1500. The audible and/or tactile indication may change or otherwise be modified as the magnitude of the difference between the two locations changes, which may provide non-visual feedback to the orthopaedic surgeon regarding how close the femoral broach 1206 is to the target depth in the femur 1500. For example, similar to the sensor module 102, the surgical navigation system 1202 may be configured to generate a series of audible beeps, the frequency of which (e.g., the activation frequency of the series of the audible beeps and/or the signal frequency of the audible beep itself) changes as the difference between the present location of the femoral broach 1206 and the reference location 1502 of the patient's femur 1500 changes. In an illustrative embodiment, for example, the frequency of a series of audible beeps may increase (i.e., the individual beeps may be generated closer in temporal proximity to each other) as the femoral broach 1206 nears the targeted depth as indicated by the reference location 1502. Similarly, the frequency of a series of tactile vibrations may increase as the femoral broach 1206 nears the reference location 1502. In this way, the orthopaedic surgeon may be updated as to the present and target locations (i.e., the present and target depths) while viewing and/or manipulating the patient's hip joint without the requirement of averting their gaze to view visual indications provided by, for example, a display.

The surgical navigation system 1202 may be embodied as any type of computer or computation device capable of tracking the location of the surgical instrument 1204 relative to a patient's boney anatomy and performing the additional functions described herein. For example, the surgical navigation system 1202 may be embodied as an operating room computer, a server, a desktop computer, a laptop computer, a tablet computer, a smartphone, a mobile computer, a smart device, a wearable computer system, or other computer or computer device. As shown in FIG. 12, the illustrative surgical navigation system 1202 includes a controller 1210, an input/output (“I/O”) subsystem 1216, a communication subsystem 1220, a tracking system 1222, a data storage 1224, and a feedback system 1226. Of course, the surgical navigation system 1202 may include additional or other components, such as those commonly found in a typical computer device or surgical navigation computer (e.g., a display, speakers, touchscreen, etc.), in other embodiments. Additionally, in some embodiments, one or more of the illustrative components may be incorporated in, or otherwise form a portion of, another component.

The controller 1210 may be embodied as any type of controller, functional block, digital logic, or other component, device, circuitry, or collection thereof capable of performing the functions described herein. In the illustrative embodiment, the controller 1210 includes a processor 1212 and a memory 1214. The processor 1212 may be embodied as any type of processor capable of performing the functions described herein. For example, the processor 1212 may be embodied as a single or multi-core processor(s), digital signal processor, microcontroller, or other processor or processing/controlling circuit. Similarly, the memory 1214 may be embodied as any type of volatile and/or non-volatile memory or data storage capable of performing the functions described herein. In operation, the memory 1214 may store various data and software used during operation of the surgical navigation system 1202 such as operating systems, applications, executable software, programs, libraries, and drivers, which may be executed or otherwise used by the processor 1212.

The controller 1210 is communicatively coupled to other components of the surgical navigation system 1202 via the I/O subsystem 1216, which may be embodied as circuitry and/or components to facilitate input/output operations between the controller 1210 (e.g., the processor 1212 and the memory 1214) and the other components of the surgical navigation system 1202. For example, the I/O subsystem 1216 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 1216 may form a portion of a system-on-a-chip (SoC) and be incorporated, along with the controller 1210 (e.g., the processor 1212 and the memory 1214) and other components of the controller 1210, on a single integrated circuit chip. Additionally, in some embodiments, the memory 1214, or portions of the memory 1214, may be incorporated into the processor 1212 of the controller 1210.

The communication subsystem 1220 may be embodied as any type of communication circuit, device, or collection thereof, capable of enabling communications between the surgical navigation system 1202 and the surgical instrument 1204. To do so, the communication subsystem 1220 may be configured to use any one or more communication technologies (e.g., wireless or wired communications) and associated protocols (e.g., Ethernet, Bluetooth®, Wi-Fi®, WiMAX, LTE, 5G, etc.) to effect such communication.

The tracking system 1222 may be embodied as any number and type of electronic components, devices, and/or associated software capable of tracking the position of the orthopaedic surgical instrument 1204 in the operative theater. To do so, the tracking system 1222 may utilize any suitable tracking technology and/or algorithm (e.g., a triangulation, trilateration, optical, etc.). For example, the tracking system 1222 may be embodied as an electrical tracking system in which the position of the orthopaedic surgical instrument 1204 is based on communications received from one or more wireless tracking sensors 1232 included in the orthopaedic surgical instrument 1204. Alternatively, in other embodiments, the tracking system 1222 may be embodied as an optical tracking system in which the position of the orthopaedic surgical instrument 1204 is based on the relative location of optical tracking sensors 1232 attached to the orthopaedic surgical instrument 1204. Regardless, the tracking system 1222 is configured to determine the present position (location and/or orientation) of the orthopaedic surgical instrument 1204 relative to the patient's bony anatomy (e.g., in a coordinate system defined by the patient's bony anatomy).

The data storage 1224 may be embodied as any type of device or devices configured for short-term and/or long-term storage of data such as, for example, solid-state drives, hard disk drives, memory devices and circuits, memory cards, non-volatile flash memory, or other data storage devices. In the illustrative embodiment, the data storage 1224 stores various data used by the surgical navigation system 1202 to perform the functions described herein. For example, the data storage 1224 may store data indicative of the present location of the surgical instrument 1204 and the reference location indicative of the target depth of the femoral broach 1206 in the patient's femur 1500.

The feedback system 1226 may be embodied as, or otherwise include, one or more non-visual indicators configured to provide a non-visual indication indicative of a difference between the present position of the femoral broach 1206 and the reference location 1502 of the patient's femur 1500. In the illustrative embodiment, the non-visual indication may have a characteristic (e.g., activation frequency, signal frequency, volume, etc.) that is dependent on a magnitude of the difference between two locations. For example, the feedback system 1226 may include one or more audible indicators (e.g., a speaker) configured to generate, when activated, an audible indication having a characteristic (e.g., activation frequency, signal frequency, volume, etc.) that is dependent on a magnitude of the difference between the present position of the femoral broach 1206 and the reference location 1502 of the patient's femur 1500. Additionally or alternatively, the feedback system 1226 may include one or more tactile indicators (e.g., a vibration motor) configured to generate, when activated, an tactile indication having a characteristic (e.g., activation frequency, signal frequency, vibration intensity, etc.) that is dependent on a magnitude of the difference between the present position of the femoral broach 1206 and the reference location 1502 of the patient's femur 1500. In some embodiments, the feedback system 1226 may also include one or more visual indicators (e.g., a light or light emitting diode) in addition to the non-visual indicators discussed above. As discussed below, the visual indicators may be used in conjunction with the non-visual indicators to provide an additional visual indication indicative of the difference between the present position of the femoral broach 1206 and the reference location 1502 of the patient's femur 1500.

The orthopaedic surgical instrument 1204 may be embodied as any type of orthopaedic surgical instrument capable of being coupled to and used with a corresponding surgical tool, the position of which is to be tracked. For example, in the illustrative embodiment, the orthopaedic surgical instrument 1204 is embodied as an automated impactor configured to operate the femoral broach 1206 to perform a reaming procedure on a proximal end of the patient's femur 1500. It should be appreciated, however, that the orthopaedic surgical instrument 1204 may be embodied as another type of manual or automated surgical instrument in other embodiments.

The orthopaedic surgical instrument 1204 includes one or more tracking sensors 1232. The tracking sensors 1232 may be embodied as any type of tracking sensors usable by the tracking system 1222 of the surgical navigation system 1202 to determine a present position of the orthopaedic surgical instrument 1204. For example, the tracking sensors 1232 may be embodied as one or more wireless sensors configured to transmit data (e.g., a beacon) to the tracking system 1222, which may determine the position of the orthopaedic surgical instrument 1204 based on the received signals (e.g., via triangulation or trilateration). Alternatively, in other embodiments, the tracking sensors 1232 may be embodied as one or more optical tracking sensors, which are usable by a camera system of the tracking system 1222 to determine the position of the orthopaedic surgical instrument 1204.

In some embodiments, the orthopaedic surgical instrument 1204 may also include the feedback system 1226 alternatively to, or in addition to, the surgical navigation system 1202. For example, the orthopaedic surgical instrument 1204 may include one or more local non-visual indicator configured to provide a non-visual indication indicative of a difference between the present position of the femoral broach 1206 and the reference location 1502 of the patient's femur 1500. In such embodiments, activation of the feedback system 1226 local to the orthopaedic surgical instrument 1204 may be controlled by the surgical navigation system 1202 and/or based on data received from the surgical navigation system 1202.

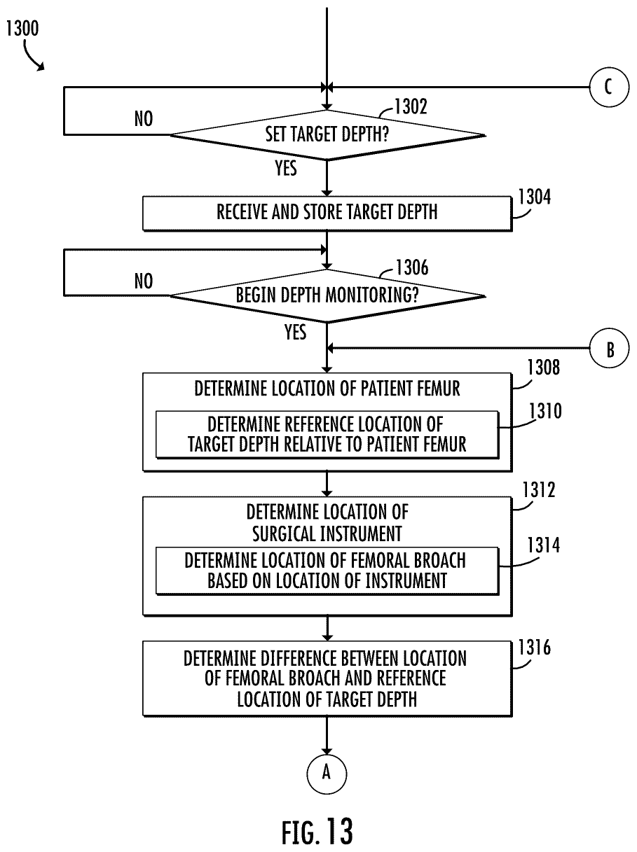

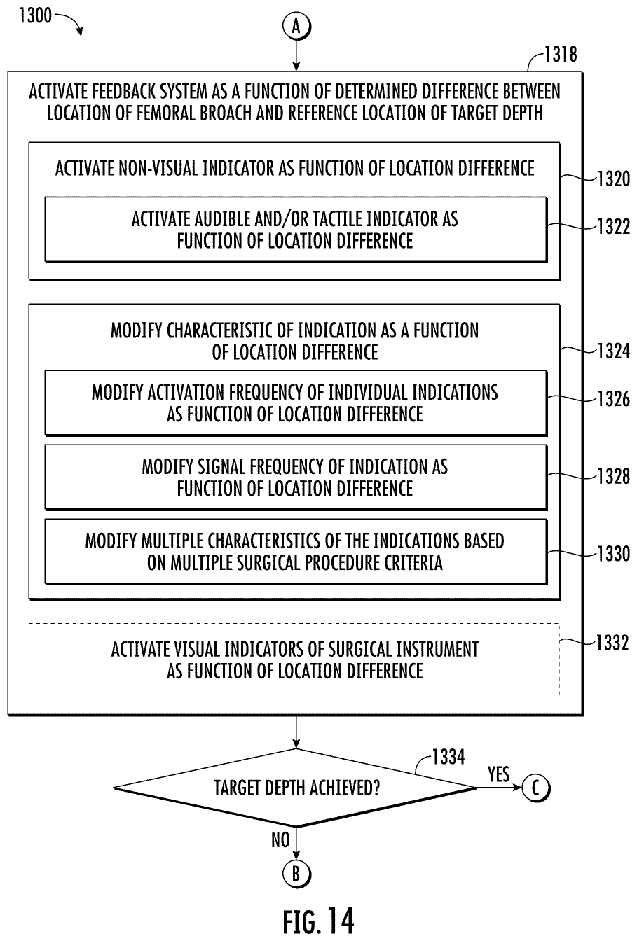

Referring now to FIGS. 13 and 14, in use, the surgical navigation system 1202 (e.g., the controller 1210) may be configured to execute a method 1300 for monitoring operation of the surgical instrument 1204 during the performance of an associated orthopaedic surgical procedure. The method 1300 begins with block 1302 in which the surgical navigation system 1202 determines whether the orthopaedic surgeon desires to set a target depth for the femoral broach 1206. If so, the method 1300 advances to block 1304 in which the surgical navigation system 1202 receives and stores the target depth provided by the orthopaedic surgeon. To do so, in some embodiments, the orthopaedic surgeon may enter a numerical number indicative of the desired depth (e.g., from the proximal end of the patient's femur 1500). Alternatively, in other embodiments, the orthopaedic surgeon may provide an indication of the desired femoral depth on a medial image of the patient's femur 1500 (e.g., an X-ray image, a computerized tomography image, a magnetic resonance imaging image, etc.). That is, the orthopaedic surgeon may annotate the reference location 1502 on the medical image to provide the desired depth to which the femoral broach 1206 should reach.

Subsequently, in block 1306, the surgical navigation system 1202 determines whether the orthopaedic surgeon desires to being monitoring the location of the surgical instrument 1204 relative to the patient's femur 1500. If so, the method 1300 advances to block 1308 in which the surgical navigation system 1202 determines the present location of the patient's femur 1500. In doing so, in block 1310, the surgical navigation system 1202 determines the reference location 1502 of the target depth relative to the present location of the patient's femur 1500.

In block 1312, the surgical navigation system 1202 determines the present location of the surgical instrument 1204 based on signals received from the tracking sensors 1232 as discussed above. It should be appreciated that the determined, present location of the surgical instrument 1204 is also indicative of the present location of the femoral broach 1206 attached to the surgical instrument 1204. As such, in block 1314, the surgical navigation system 1202 determines the present location of the femoral broach 1206 based on the determined present location of the surgical instrument 1204. Additionally, it should be appreciated that the determined positions of the surgical instrument 1204, femoral broach 1206, and the femur 1500 are within the same coordinate system.

After the surgical navigation system 1202 has determined the present location of the patient's femur 1500 in block 1308 and the present location of the surgical instrument 1204 in block 1312, the surgical navigation system 1202 determines a difference between the present location of the femoral broach 1206 and the reference location 1502 of the target depth of the femoral broach 1206 in the femur 1500 of the patient in block 1316. To do so, the surgical navigation system 1202 may compare the present location of the femoral broach 1206 and the reference location 1502 to determine a magnitude of the difference between the two locations. For example, FIG. 15 illustrates the impaction of the femoral broach 1206 into the patient's femur 1500 at an early stage of the procedure at which the femoral broach 1206 is not yet at the reference location 1502 indicative of the target depth. Comparatively, FIG. 16 illustrates the femoral broach 1206 having been fulling impacted into the patient's femur 1500 to the target depth 1502.

Subsequently, in block 1318 of FIG. 14, the surgical navigation system 1202 activates the feedback system 1226 as a function of the determined difference between the determined present location of the femoral broach 1206 and the determined reference location 1502 of the target depth of the femoral broach 1206 in the femur 1500 of the patient. In particular, in block 1320, the surgical navigation system 1202 may activate the non-visual indicators as a function of the determined difference between the two locations. For example, in block 1322, the surgical navigation system 1202 may activate the audible indicator(s) and/or the tactile indicator(s) of the feedback system 1226 as a function of the difference between the determined present location of the femoral broach 1206 and the determined reference location 1502 of the target depth of the femoral broach 1206. In doing so, a characteristic of the non-visual indication provided by the non-visual indicator(s) may be dependent on a magnitude of the difference between the two locations. For example, the activation frequency (i.e., the temporal spacing) of a series of non-visual indications and/or the signal frequency of the non-visual indication may be dependent on the magnitude of the determined difference. In this way, if the magnitude of the determined difference is relatively large, the activation frequency of the non-visual indications may be low (e.g., individual audible “beeps” may be spaced apart by a larger amount of time) and/or the signal frequency of the non-visual indication may be low (e.g., the audible “beep” may be generated at a low signal frequency). Conversely, if the magnitude of the determined difference is relatively small, the activation frequency of the non-visual indications may be high (e.g., individual audible “beeps” may be close together in time) and/or the signal frequency of the non-visual indication may be high (e.g., the audible “beep” may be generated at a high signal frequency). In this way, the orthopaedic surgeon receives non-visual feedback as a notification that the femoral broach 1206 is approaching or moving away from the target depth of the patient's femur 1500, without the requirement to view displayed force values.

Additionally, in block 1324, the surgical navigation system 1202 may change or modify the characteristic(s) of the non-visual indication as a function of or based on the magnitude of the difference between the determined present location of the femoral broach 1206 and the determined reference location 1502 of the target depth of the femoral broach 1206. That is, as the magnitude changes, the surgical navigation system 1202 may likewise modify or change the characteristic. For example in block 1326, the surgical navigation system 1202 may modify the activation frequency of individual indications as a function of the magnitude of the determined difference (e.g., increase the activation frequency of audible “beeps” in response to the magnitude decreasing). Additionally or alternatively, in block 1328, the surgical navigation system 1202 may modify the signal frequency of the indication as a function of the magnitude of the determined difference (e.g., increase the signal frequency of the audible “beep” in response to the magnitude decreasing).

In some embodiments, in block 1330, the surgical navigation system 1202 may modify multiple characteristics of the non-visual indications based on multiple surgical procedure criteria. For example, the activation frequency of the non-visual indication may correspond to a difference in location of the femoral broach 1206 and the target depth and the signal frequency of the non-visual indication may correspond to a presently determined femoral inclination or anteversion and a targe inclination or anteversion of the femoral broach 1206 (and, thereby, a femoral prosthesis). In this way, the orthopaedic surgeon may monitor and adjust multiple implant alignment characteristics contemporaneously with each other.

In some embodiments, in block 1332, the surgical navigation system 1202 may also activate one or more visual indicators of the feedback system 1226 as a function of the determined location difference, in addition to the non-visual indicators. Regardless, after the surgical navigation system 1202 has activated the feedback system 1226 in block 1318, the method 1300 advances to block 1334. In block 1334, the surgical navigation system 1202 determines if the orthopaedic surgeon has achieved the target depth (and/or other surgical procedure criteria) has been achieved. If so, the method 1300 loops back to block 1302 in which the surgical navigation system 1202 awaits for a new target depth to be set. Alternatively, if the target depth has not yet been achieved, the method 1300 loops back to block 1308 in which the surgical navigation system 1202 continues to determine the present location so the patient's femur 1500 and the surgical instrument 1204.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. An orthopaedic surgical device comprising:
a tibial paddle shaped to be positioned between a proximal tibia and distal femur of a patient's knee joint;
a sensor array positioned in the tibial paddle, the sensor array including a plurality of sensors configured to generate sensor data indicative of a force between the patient's tibia and femur;
a non-visual indicator; and
a controller configured to:
receive the sensor data from the sensor array,
determine a difference between the sensor data and a target force value, and
activate the non-visual indicator to produce a non-visual indication having a characteristic that is dependent on a magnitude of the difference between the sensor data and the target force value.

2. The orthopaedic surgical device of claim 1, wherein to receive sensor data from the sensor array comprises to receive sensor data indicative of a force on a medial or a lateral side of the patient's knee joint, and
wherein the target force value is equal to a previously measured force value of the other one of the medial or the lateral side of the patient's knee joint.

3. The orthopaedic surgical device of claim 1, wherein the non-visual indicator comprises an audible indicator, and
wherein to activate the non-visual indicator comprises to activate the audible indicator to produce an audible indication having a characteristic that is dependent on the magnitude of the difference between the sensor data and the target force value.

4. The orthopaedic surgical device of claim 1, wherein the non-visual indicator comprises a tactile indicator, and
wherein to activate the non-visual indicator comprises to activate the tactile indicator to produce an tactile indication having a characteristic that is dependent on the magnitude of the difference between the sensor data and the target force value.

5. The orthopaedic surgical device of claim 1, wherein to activate the non-visual indicator comprises to modify the characteristic of the non-visual indication as a function of the magnitude of the difference between the sensor data and the target force value.

6. The orthopaedic surgical device of claim 5, wherein the non-visual indication comprises a plurality of individual indications that are produced at an activation frequency, and
wherein to modify the characteristic of the non-visual indication comprises to modify the activation frequency of the plurality of individual indications.

7. The orthopaedic surgical device of claim 6, wherein the non-visual indicator comprises an audible indicator configured to produce, when activated, a plurality of individual audible indications at the activation frequency.

8. The orthopaedic surgical device of claim 5, wherein the non-visual indication comprises a non-visual indication having a signal frequency, and
wherein to modify the characteristic of the non-visual indication comprises to modify the signal frequency of the non-visual indication.

9. The orthopaedic surgical device of claim 8, wherein the non-visual indicator comprises an audible indicator configured to produce, when activated, an audible indication having the signal frequency.

10. The orthopaedic surgical device of claim 1, wherein to determine the difference between the sensor data and the target force value comprises to determine a difference between the sensor data and each of a plurality of target force values, and
wherein to activate the non-visual indicator comprises to activate the non-visual indicator to produce a non-visual indication having multiple characteristics, each of which is dependent on a magnitude of the difference between the sensor data and a different one of the plurality of target force values.

11. A method for monitoring joint force of a patient's knee joint during performance of an orthopaedic surgical procedure, the method comprising:
determining, by a sensor module located between a proximal tibia and a distal femur of the patient's knee joint, sensor data indicative of a force between the patient's tibia and femur;
determining, by the sensor module, a difference between the sensor data and a target force value; and
activating, by the sensor module, a non-visual indicator of the sensor module as a function of the difference between the sensor data and the target force value, wherein the non-visual indicator, when activated, produces a non-visual indication having a characteristic that is dependent on a magnitude of the difference between the sensor data and the target force value.

12. The method of claim 11, wherein determining sensor data indicative of the force between the patient's tibia and femur comprises determining sensor data indicative of a force on a medial or a lateral side of the patient's knee joint, and
wherein the target force value is equal to a previously measured force value of the other one of the medial or the lateral side of the patient's knee joint.

13. The method of claim 11, wherein activating the non-visual indicator comprises activating an audible indicator as a function of the difference between the sensor data and the target force value.

14. The method of claim 11, wherein activating the non-visual indicator comprises activating a tactile indicator as a function of the difference between the sensor data and the target force value.

15. The method of claim 11, wherein activating the non-visual indicator of the sensor module comprises modifying the characteristic of the non-visual indication as a function of the magnitude of the difference between the sensor data and the target force value.

16. The method of claim 15, wherein the non-visual indication comprises a plurality of individual indications that are produced at an activation frequency, and
wherein modifying the characteristic of the non-visual indication comprises modifying the activation frequency of the plurality of individual indications.

17. The method of claim 15, wherein the non-visual indication comprises a non-visual indication have a signal frequency, and
wherein modifying the characteristic of the non-visual indication comprises modifying the signal frequency of the non-visual indication.

18. The method of claim 11, wherein determining the difference between the sensor data and the target force value comprises determining a difference between the sensor data and each of a plurality of target force values, and
wherein activating the non-visual indicator comprises activating the non-visual indicator as a function of the differences between the sensor data and the plurality of target force values, wherein the non-visual indicator, when activated, produces a non-visual indication having multiple characteristics, each of which is dependent on a magnitude of the difference between the sensor data and a different one of the plurality of target force values.

19. A surgical navigation system comprising:
a tracking system configured to determine a present location of a surgical instrument relative to a femur of a patient;
a non-visual indicator; and
a controller configured to
determine a present location of a femoral broach attached to the surgical instrument based on the location of the surgical instrument;
determine a difference between the present location of the femoral broach and a reference location indicative of a target depth of the femoral broach in the femur of the patient; and
activate the non-visual indicator to produce a non-visual indication having a characteristic that is dependent on a magnitude of the difference between the location of the femoral broach and the reference location of the target depth of the femoral broach in the femur of the patient.

20. The surgical navigation system of claim 19, wherein the controller is further configured to:
determine a present alignment of the femoral broach relative to the femur of the patient based on the location of the surgical instrument; and
determine a difference between the present alignment of the femoral broach and a reference alignment,
wherein to activate the non-visual indicator comprises to activate the non-visual indicator to produce a non-visual indication having multiple characteristics, wherein (i) a first characteristic of the multiple characteristics is dependent on the magnitude of the difference between the location of the femoral broach and the reference location of the target depth of the femoral broach in the femur of the patient and (ii) a second characteristic of the multiple characteristics is dependent on the magnitude of the difference between the present alignment of the femoral broach and a reference alignment, wherein the reference alignment comprises a target amount of femoral anteversion or femoral inclination.
